(19) United States
(12) Patent Application Publication
(10) Pub. No.: US 2026/0306167 A1
(43) Pub. Date: Oct. 8, 2026

(54) ULTRASOUND TISSUE TREATMENT APPARATUS AND METHOD

(71) Applicant: HEALIUM MEDICAL LTD., Yokneam (IL)

(72) Inventors: Ran Sela, Ramat Hasharon (IL); Yuri Megel, Nesher (IL); Assaf Rubissa, Yaad (IL); Shai Fisher, Haifa (IL); Amir Weizman, Tsur Yitzhak (IL)

(21) Appl. No.: 19/482,513

(22) PCT Filed: May 8, 2024
(86) PCT No.: PCT/IB2024/054483

§ 371 Date: Nov. 7, 2025

Related U.S. Application Data
(60) Provisional application No. 63/464,722, filed on May 8, 2023.

(51) Int. Cl. A61B 18/14; A61B 18/00

(52) U.S. Cl. CPC A61B 18/1492; A61B 2018/00208; A61B 2018/00351; A61B 2018/00577; A61B 2018/00994

(57) ABSTRACT

An apparatus is provided (20) including a catheter (40) having a rotatable element (50) and a handle (25), at distal and proximal ends of the catheter, respectively. The handle has a cylindrical drum (42), and a drive cable (43) extending distally from the cylindrical drum to the rotatable element, the drive cable transmits rotational motion from the cylindrical drum to the rotatable element. A handle shaft (49) is disposed within the handle and extends proximally from a proximal end of the cylindrical drum. A motor unit includes a motor (28) and a motor-unit shaft (47) extending from the motor, the motor-unit shaft being configured to be reversibly couplable to the handle shaft, such that the motor unit shaft transmits rotational motion from the motor to the cylindrical drum. Other applications are also described.

### CROSS-REFERENCES TO RELATED APPLICATIONS

The present application claims priority from U.S. Provisional Patent Application No. 63/464,722 to Sela et al., filed May 8, 2023, entitled “ULTRASOUND TISSUE TREATMENT APPARATUS AND METHOD”, which is incorporated herein by reference.

### FIELD OF EMBODIMENTS OF THE INVENTION

Some applications of the present invention generally relate to devices and methods for treatment of tissue by application of energy thereto, and more particularly to ablation of cardiac tissue by application of ultrasound energy for treating cardiac conditions, such as but not limited to cardiac arrhythmias.

### BACKGROUND

Atrial fibrillation is a common cardiac arrhythmia involving the atria of the heart. During atrial fibrillation, the atria beat irregularly and out of coordination with the ventricles of the heart, thereby disrupting efficient beating of the heart. Atrial fibrillation symptoms often include heart palpitations, shortness of breath and weakness. A major concern with atrial fibrillation is the potential to develop blood clots within the atria of the heart. These blood clots forming in the heart may circulate to other organs and lead to serious medical conditions such as strokes.

Atrial fibrillation is generally caused by abnormal electrical activity in the heart. During atrial fibrillation, electrical discharges may be generated by parts of the atria which do not normally generate electrical discharges, such as pulmonary vein ostia in the atrium.

Ablation procedures are generally used to terminate a faulty electrical pathway from sections of the heart, especially in those who are prone to developing cardiac arrhythmias and to restore the heart to its normal rhythm. For example, pulmonary vein isolation by ablation is a common medical procedure for treatment of atrial fibrillation.

### SUMMARY

In some applications of the present invention, apparatus and methods are provided for treatment of tissue by application of energy thereto. Typically, an energy source, e.g., an ultrasound transducer coupled to a shaft of a transluminal delivery tool, e.g., a catheter, is positioned within a lumen in a body of a subject and is activated to apply treatment energy in order to treat tissue surrounding the lumen. In particular, there is provided in accordance with some applications of the present invention, apparatus for use with a lumen, e.g., a pulmonary vein that extends from a heart chamber, e.g., the left atrium of the heart. Typically, the apparatus applies ultrasound energy to ablate tissue of the ostium of the pulmonary vein to electrically isolate the pulmonary vein to treat cardiac arrhythmia. For some applications, the apparatus comprises a catheter comprising at least one ultrasound transducer coupled to a distal end of the catheter and configured to be inserted into the chamber of the subject's heart, and to ablate the tissue of the ostium of the pulmonary vein by application of ultrasound energy. Additionally, or alternatively to applying ablative ultrasound energy, the ultrasound transducer is configured to image tissue of the subject by applying non-ablative ultrasound energy. Typically, the apparatus described herein is configured to allow both rotational motion and axial back and forth motion of the ultrasound transducer. Motion of the ultrasound transducer typically facilitates both imaging of tissue and also enhanced targeting of areas in the tissue designated for ablation. In accordance with some applications of the present invention, the apparatus allows for both manual and motorized motion of the rotatable ultrasound transducer.

For some such applications, the catheter comprises, in addition to the rotatable ultrasound transducer coupled to the distal end of catheter, a handle at the proximal end of the catheter. For some applications, the handle includes a cylindrical drum configured to be rotated by a user. A drive cable extends distally from the cylindrical drum to the rotatable ultrasound transducer at the distal end of the catheter, the drive cable being configured to transmit rotational motion from the cylindrical drum to the rotatable ultrasound transducer (such that rotation of the cylindrical drum by the user causes rotation of ultrasound transducer). Additionally, a handle shaft is disposed within the handle, the handle shaft extending proximally from a proximal end of the cylindrical drum. The apparatus additionally comprises a motor unit comprising a motor, and a motor-unit shaft extending from the motor. The motor-unit shaft is reversibly couplable to the handle shaft, such that the motor unit shaft transmits rotational motion from the motor to the cylindrical drum, when the motor-unit shaft is coupled to the handle shaft (such that rotation of the cylindrical drum by the motor causes rotation of the ultrasound transducer).

Typically, the apparatus comprises a snap-coupling mechanism, which is configured to reversibly couple the motor-unit shaft and the handle shaft such that the motor unit shaft transmits rotational motion and axial motion from the motor to the handle shaft. The motor couples the motor-unit shaft and the handle shaft to each other by advancing a distal end of the motor-unit shaft distally within the handle and decouples the motor-unit shaft and the handle shaft from each other by retracting the distal end of the motor-unit shaft proximally within the handle. In such a manner, the motor-unit shaft is reversibly couplable to the handle shaft to allow motorized rotation of the ultrasound transducer when the motor-unit shaft is coupled to the handle shaft, and manual rotation of the ultrasound transducer when the motor-unit shaft is not coupled to the handle shaft.

As described hereinabove, the ultrasound transducer is a rotatable ultrasound transducer, typically configured to apply a treatment to (and/or image tissue of) the subject when disposed at a plurality of different rotational positions. In some cases, static friction is caused while the ultrasound transducer undergoes rotational movements between the plurality of different rotational positions. In accordance with some applications of the present invention, in order to reduce static friction acting upon the rotatable ultrasound transducer between the rotational movements, the rotational ultrasound transducer is typically driven to continuously move axially between the rotational movements of the rotational ultrasound transducer.

For some applications, the apparatus comprises sensor mechanisms configured to detect axial and/or rotational positions of the ultrasound transducer. For example, the apparatus comprises a magnetic sensor mechanism and a computer processor operatable with the apparatus, which is configured to derive axial and/or rotational positions of the ultrasound transducer based upon the magnetic sensor mechanism. Typically, as described hereinabove, the apparatus comprises a drive cable extending distally from within the handle to the ultrasound transducer at the distal end of the catheter, the drive cable being configured to transmit rotational and axial motion from within the handle to the ultrasound transducer. For some applications, a stationary tube is disposed around the drive cable, the tube being configured to remain stationary as the drive cable undergoes rotational and axial motion. A plurality of magnetic rods is disposed at respective axial and rotational locations within a given region of the stationary tube, each of the magnetic rods being configured to generate magnetic fields having respective, different characteristics from each other. A magnetic sensor is coupled to the drive cable and configured to detect magnetic fields generated by the magnetic rods, and a computer processor is typically configured to receive a signal generated by the magnetic sensor and derive axial and rotational positions of the ultrasound sensor based upon the signal.

For some applications, the transluminal ablation catheter comprises more than one, e.g., two ablative ultrasound transducers that are arranged with respect to each other and/or to the shaft of the transluminal catheter to create a convergent ultrasound beam for focusing at the target tissue (e.g., the tissue of the pulmonary vein ostium). For some applications, a sensing ultrasound transducer is positioned between the ablative ultrasound transducers. For example, the transluminal ablation catheter comprises two ablative ultrasound transducers, each of the ablative ultrasound transducers being disposed on a side of the catheter shaft at an angle of between 8 degrees and 15 degrees from an axis of the shaft, such as to create a convergent ultrasound beam. For some such applications, an imaging ultrasound transducer is disposed between the two ablative ultrasound transducers and configured to acquire one or more ultrasonic images of the tissue of the ostium.

As described hereinabove, the transluminal ablation catheter comprises a rotatable ultrasound transducer at the distal end of the catheter. The catheter typically comprises fluid lumens for delivering fluid to the vicinity of the rotatable ultrasound transducer for cooling of the ultrasound transducer. For some such applications, the transluminal ablation catheter comprises a fluid lumen rotational manifold configured to prevent rotation of the fluid lumens while the ultrasound transducer rotates, thereby preventing twisting and tangling of the fluid lumens. Typically, the drive cable that extends distally from within the handle to the rotatable ultrasound transducer (the drive cable transmits rotational motion from within the handle to the rotatable ultrasound transducer), defines a drive-cable fluid inflow lumen and a drive-cable fluid outflow lumen for cooling of the rotatable ultrasound transducer. The fluid manifold typically includes a fluid entry port, a fluid exit port, a rotationally static portion that contains a rotationally-static fluid inflow lumen, which is in fluid communication with the fluid entry port, and a rotationally-static fluid outflow lumen, which is in fluid communication with the fluid exit port. The fluid manifold additionally includes a rotational portion that is configured to rotate with the drive cable, and defines (a) a rotational fluid inflow lumen that is in fluid communication with both the rotationally-static fluid inflow lumen and the drive-cable fluid inflow lumen, thereby placing the rotationally-static fluid inflow lumen and the drive-cable fluid inflow lumen in fluid communication with each other; and (b) a rotational fluid outflow lumen that is in fluid communication with the rotationally-static fluid outflow lumen and the drive-cable fluid outflow lumen, thereby placing the rotationally-static fluid outflow lumen and the drive-cable fluid outflow lumen in fluid communication with each other.

There is therefore provided, in accordance with some applications of the present invention, an apparatus including:
a catheter having a distal end and a proximal end, the catheter including:
   a rotatable element at the distal end of the catheter;
   a handle at the proximal end of the catheter, the handle including a cylindrical drum;
   a drive cable extending distally from the cylindrical drum to the rotatable element at the distal end of the catheter, the drive cable being configured to transmit rotational motion from the cylindrical drum to the rotatable element; and
   a handle shaft disposed within the handle, the handle shaft extending proximally from a proximal end of the cylindrical drum; and
a motor unit including:
   a motor; and
   a motor-unit shaft extending from the motor, the motor-unit shaft being configured to be reversibly couplable to the handle shaft, such that the motor unit shaft transmits rotational motion from the motor to the cylindrical drum.

In some applications, the handle defines one or more windows and the cylindrical drum configured to be manually rotated directly via the one or more windows.

In some applications, the apparatus further includes a user-operated rotation shaft configured to be couplable to the handle shaft, such as to facilitate manual rotation of the cylindrical drum, via the user-operated rotation shaft and the handle shaft.

In some applications, the catheter includes a transluminal ablation catheter.

In some applications, the catheter includes one or more sensors configured to detect a rotational position of the rotatable element.

In some applications, the rotatable element includes a rotatable ultrasound transducer.

In some applications, the rotatable ultrasound transducer is configured to be inserted into a left atrium in a vicinity of a pulmonary vein ostium and is configured to ablate tissue of the pulmonary vein ostium, to thereby electrically isolate the pulmonary vein.

In some applications, the rotatable ultrasound transducer is configured to be inserted into a coronary sinus in a vicinity of a mitral isthmus and is configured to ablate tissue of the isthmus.

In some applications, the motor-unit shaft and the handle shaft include a snap-coupling mechanism, which is configured to reversibly couple the motor-unit shaft and the handle shaft such that the motor unit shaft transmits rotational motion and axial motion from the motor to the handle shaft.

In some applications, the motor is configured to:
couple the motor-unit shaft and the handle shaft to each other by advancing a distal end of the motor-unit shaft distally within the handle, and
decouple the motor-unit shaft and the handle shaft from each other by retracting the distal end of the motor-unit shaft proximally within the handle.

There is further provided, in accordance with some applications of the present invention, an apparatus including:
a catheter having a distal end and a proximal end, the catheter including:
   a rotatable element at the distal end of the catheter;
   a handle at the proximal end of the catheter;
   a drive cable extending distally from within the handle to the rotatable element at the distal end of the catheter, the drive cable being configured to transmit rotational motion from within the handle to the rotatable element; and
   a handle shaft disposed within the handle, the handle shaft being coupled to the drive cable; and
a motor unit including:
   a motor; and
   a motor-unit shaft extending from the motor,
      the motor-unit shaft and the handle shaft including a snap-coupling mechanism, which is configured to reversibly couple the motor-unit shaft and the handle shaft such that the motor unit shaft transmits rotational motion and axial motion from the motor to the handle shaft, and
      the motor being configured to:
         couple the motor-unit shaft and the handle shaft to each other by advancing a distal end of the motor-unit shaft distally within the handle, and
         decouple the motor-unit shaft and the handle shaft from each other by retracting the distal end of the motor-unit shaft proximally within the handle.

In some applications, the catheter includes a transluminal ablation catheter.

In some applications, the rotatable element includes a rotatable ultrasound transducer.

In some applications, the rotatable ultrasound transducer is configured to be inserted into a left atrium in a vicinity of a pulmonary vein ostium and is configured to ablate tissue of the pulmonary vein ostium, to thereby electrically isolate the pulmonary vein.

In some applications, the rotatable ultrasound transducer is configured to be inserted into a coronary sinus in a vicinity of a mitral isthmus and is configured to ablate tissue of the isthmus.

In some applications, the apparatus further includes a user-operated rotation shaft configured to be couplable to the handle shaft, such as to facilitate manual rotation of the handle shaft.

In some applications, the user-operated rotation shaft and the handle shaft including a snap-coupling mechanism, which is configured to reversibly couple the motor-unit shaft and the handle shaft such that the motor unit shaft transmits rotational motion and axial motion from the motor to the handle shaft.

There is further provided, in accordance with some applications of the present invention, an apparatus including:
a catheter having a distal end and a proximal end, the catheter including:
   a rotatable element at the distal end of the catheter configured to apply a treatment to a subject when disposed at a plurality of different rotational positions;
   a handle at the proximal end of the catheter; and
   a drive cable extending distally from within the handle to the rotatable element at the distal end of the catheter, the drive cable being configured to transmit rotational motion from within the handle to the rotatable element; and
a motor configured, during the treatment, to drive the rotatable element to undergo rotational movements between the plurality of different rotational positions, and to reduce static friction acting upon the rotatable element between the rotational movements, by driving the rotatable element to continuously move axially between the rotational movements of the rotatable element.

In some application , the catheter includes a transluminal ablation catheter.

In some applications, the motor is configured to drive the rotatable element to continuously move axially between the rotational movements of the rotatable element, to move in an axial back and forth motion to prevent the rotatable element from being susceptible to static friction between the rotational movements of the rotatable element.

In some applications, the rotatable element includes a rotatable ultrasound transducer.

In some applications, the rotatable ultrasound transducer is configured to be inserted into a left atrium in a vicinity of a pulmonary vein ostium and is configured to ablate tissue of the pulmonary vein ostium, to thereby electrically isolate the pulmonary vein.

In some applications, the rotatable ultrasound transducer is configured to be inserted into a coronary sinus in a vicinity of a mitral isthmus and is configured to ablate tissue of the isthmus.

There is further provided, in accordance with some applications of the present invention, an apparatus for use with a fluid, the apparatus including:
a catheter having a distal end and a proximal end, the catheter including:
   a rotatable element at the distal end of the catheter configured to apply a treatment to a subject when disposed at a plurality of different rotational positions;
   a handle at the proximal end of the catheter; and
   a drive cable extending distally from within the handle to the rotatable element at the distal end of the catheter, the drive cable being configured to transmit rotational motion from within the handle to the rotatable element, the drive-cable defining a drive-cable fluid inflow lumen and a drive-cable fluid outflow lumen; and
a fluid manifold, including:
   a fluid entry port;
   a fluid exit port;
   a rotationally static portion that contains a rotationally-static fluid inflow lumen, which is in fluid communication with the fluid entry port, and a rotationally-static fluid outflow lumen, which is in fluid communication with the fluid exit port; and
   a rotational portion that is configured to rotate with the drive cable, the rotational portion defining:
   a rotational fluid inflow lumen that is in fluid communication with both the rotationally-static fluid inflow lumen and the drive-cable fluid inflow lumen, thereby placing the rotationally-static fluid inflow lumen and the drive-cable fluid inflow lumen in fluid communication with each other; and
   a rotational fluid outflow lumen that is in fluid communication with the rotationally-static fluid outflow lumen and the drive-cable fluid outflow lumen, thereby placing the rotationally-static fluid outflow lumen and the drive-cable fluid outflow lumen in fluid communication with each other.

In some applications, the catheter includes a transluminal ablation catheter.

In some applications, the rotatable element includes a rotatable ultrasound transducer. In some applications, the rotatable ultrasound transducer is configured to be inserted into a left atrium in a vicinity of a pulmonary vein ostium and is configured to ablate tissue of the pulmonary vein ostium, to thereby electrically isolate the pulmonary vein.

In some applications, the rotatable ultrasound transducer is configured to be inserted into a coronary sinus in a vicinity of a mitral isthmus and is configured to ablate tissue of the isthmus.

There is further provided, in accordance with some applications of the present invention, an apparatus including:
a catheter having a distal end and a proximal end, the catheter including:
   an ultrasound transducer at the distal end of the catheter configured to apply an ablative ultrasound treatment within a lumen of a subject;
   a handle at the proximal end of the catheter;
   a drive cable extending distally from within the handle to the ultrasound transducer at the distal end of the catheter, the drive cable being configured to transmit rotational and axial motion from within the handle to the ultrasound transducer;
   a stationary tube disposed around the drive cable, the tube being configured to remain stationary as the drive cable undergoes rotational and axial motion;
   a plurality of magnetic rods disposed a respective axial and rotational locations within a given region of the stationary tube, each of the rods being configured to generate magnetic fields having respective, different characteristics from each other;
   a magnetic sensor coupled to the drive cable and configured to detect magnetic fields generated by the magnetic rods; and
   a computer processor configured to receive a signal generated by the magnetic sensor and derive axial and rotational positions of the ultrasound transducer based upon the signal.

In some applications, the ultrasound transducer is configured to be inserted into a left atrium in a vicinity of a pulmonary vein ostium and is configured to ablate tissue of the pulmonary vein ostium, to thereby electrically isolate the pulmonary vein.

In some applications, the ultrasound transducer is configured to be inserted into a coronary sinus in a vicinity of a mitral isthmus and is configured to ablate tissue of the isthmus.

In some applications, the plurality of magnetic rods vary in length.

In some applications, the plurality of magnetic rods vary in magnetic properties.

There is further provided, in accordance with some applications of the present invention, apparatus for use with a lumen of a subject that extends from a chamber of a heart of the subject, the apparatus including:
a transluminal ablation catheter a distal portion of which is configured to be positioned inside the subject's lumen, the transluminal ablation catheter including:
   a shaft;
   two ablative ultrasound transducers configured to be inserted into the chamber of the subject's heart, and to ablate tissue of an ostium of the lumen by applying ablative ultrasound energy to the tissue of the ostium, each of the ablative ultrasound transducers being disposed on a side of the shaft at an angle of between 8 degrees and 15 degrees from an axis of the shaft, such as to create a convergent ultrasound beam; and
   an imaging ultrasound transducer disposed between the two ablative ultrasound transducers and configured to acquire one or more ultrasonic images of the tissue of the ostium.

In some applications, each of the ablative ultrasound transducers is disposed on a side of the shaft at an angle of between 11 degrees and 12 degrees from an axis of the shaft, such as to create a convergent ultrasound beam.

There is still further provided in accordance with some applications of the present invention, apparatus for use with tissue of a subject, the apparatus including:
a transluminal ablation catheter including:
   at least one ultrasound transducer configured to be inserted into a lumen of the subject's heart to ablate tissue surrounding the lumen by applying ultrasound energy to the tissue; and
   a cylindrically-shaped expandable element configured to be disposed around the at least one ultrasound transducer and to temporarily anchor a distal portion of the transluminal ablation catheter in the lumen by the expandable element contacting a wall of the lumen, the expandable element comprising a compliant material that is configured to be expanded to a diameter that is in a range of 4 mm to 30 mm.

For some applications, the lumen includes a lumen that extends from a chamber of the subject's heart, and the tissue includes tissue of an ostium of the lumen, and
the ultrasound transducer is configured to ablate a circular lesion around the ostium of the lumen by rotating within the expandable element while applying the ultrasound energy to the tissue, and
the expandable element is configured to be expanded to a diameter of 8 mm to 30 mm to temporarily anchor the catheter in the lumen.

For some applications, the lumen includes a lumen of a coronary sinus of the subject, and the tissue includes cardiac tissue, and
the ultrasound transducer is configured to ablate a linear lesion in the tissue by applying the ultrasound energy from within the coronary sinus; and
the expandable element is configured to be expanded to a diameter of 4 mm to 10 mm to temporarily anchor the catheter in the coronary sinus.

For some applications, the expandable element comprises a compliant balloon that is configured to undergo 400% elongation along its circumferential direction, such that the balloon is highly compliant as it expands radially.

For some applications, the expandable element is configured to facilitate formation of a linear lesion within the lumen, by allowing the ultrasound transducer to be moved axially within the expandable element while the expandable element is in an axially fixed in position with respect to the wall of the lumen and the ultrasound transducer is applying the ultrasound energy towards the tissue.

For some applications, the expandable element is configured to facilitate formation of a linear lesion within the lumen, by allowing the transluminal ablation catheter to be pulled along the lumen with the expandable element in a partially expanded state, while the ultrasound transducer is applying the ultrasound energy towards the tissue.

The present invention will be more fully understood from the following detailed description of embodiments thereof, taken together with the drawings, in which:

### BRIEF DESCRIPTION OF THE DRAWINGS

FIG. 1 is a schematic illustration of an apparatus for application of ultrasound energy to tissue within a body of a subject, in accordance with some applications of the present invention;

FIGS. 2A and 2B are schematic illustrations of a handle of the apparatus for application of ultrasound energy to tissue within a body of a subject, in accordance with some applications of the present invention;

FIG. 3 is a schematic illustration of components of the handle of the apparatus for application of ultrasound energy to tissue within a body of a subject, in accordance with some applications of the present invention;

FIGS. 4A, 4B, and 4C are schematic illustrations of the engagement of a motor-coupling mechanism configured to reversibly couple a motor to the handle of the apparatus to transmit rotational motion and axial motion from the motor to the ultrasound transducer, in accordance with some applications of the present invention;

FIG. 5 is a schematic illustration of the disengagement of a motor-coupling mechanism, in accordance with some applications of the present invention;

FIGS. 6A and 6B are schematic illustrations of a manual handle for coupling to the handle of the apparatus to transmit rotational motion and axial motion from a human operator to the ultrasound transducer, in accordance with some applications of the present invention;

FIGS. 7A and 7B are schematic illustrations of a configuration of a plurality of ultrasound transducers in the apparatus for application of ultrasound energy to tissue within a body of a subject, in accordance with some applications of the present invention;

FIG. 7C is a graph showing the axial distance from the ultrasound transducers of FIGS. 7A and 7B at which the beams from the ultrasound transducers of FIGS. 7A and 7B cross as a function of the tilt angle of the ultrasound transducers, in accordance with some applications of the present invention;

FIG. 7D is a graph showing the beam shape and axial distance from the ultrasound transducers at which the beams cross for the respective tilt angles of the ultrasound transducers shown in FIG. 7C, in accordance with some applications of the present invention;

FIGS. 8A and 8B are schematic illustrations of a mechanism configure to determine the rotational and axial position of the ultrasound transducer, in accordance with some applications of the present invention;

FIGS. 9A and 9B are schematic illustrations of a fluid lumen rotational manifold for use in the apparatus for application of ultrasound energy to tissue within a body of a subject, in accordance with some applications of the present invention;

FIG. 10 is a schematic illustration of apparatus for application of ultrasound energy to tissue within a body of a subject, in accordance with some applications of the present invention; and

FIGS. 11A and 11B are schematic illustrations of an expandable element of the apparatus for application of ultrasound energy to tissue within a body of a subject, in accordance with some applications of the present invention.

### DETAILED DESCRIPTION OF EMBODIMENTS

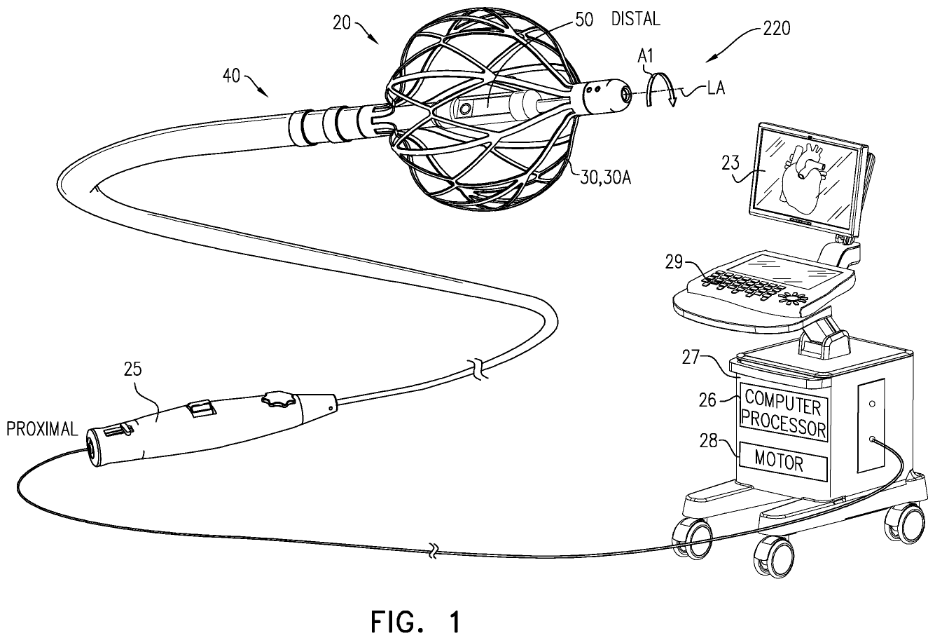

Reference is now made to FIG. 1A, which shows an overview of system 220 for ultrasound tissue treatment, including a control console 27, and apparatus 20 for application of ultrasound energy to tissue within the body of the subject. Apparatus 20 typically comprises a catheter 40 including at least one ultrasound transducer 50 configured to apply ultrasound energy toward a target tissue, and a handle 25. Apparatus 20 is operatable with control console 27, which typically includes a computer processor 26, motor 28 and a display 23.

Computer processor 26 is configured to detect various parameters related to the position and orientation of the ultrasound transducer and/or parameters related to application of the ultrasound energy (such as parameters of applied and/or reflected ultrasound energy, etc.), and to drive the ultrasound transducer to transmit the ultrasound energy (e.g., by selecting optimal ultrasound energy application parameters, etc.), as will be described in further detail hereinbelow. For some applications, computer processor 26 drives the ultrasound transducer to rotate and/or translate back and forth, as will be described hereinbelow. For example, the computer processor may control motion of the ultrasound transducer through an actuator (e.g., motor 28) housed in the control console (or elsewhere in system 220 such as handle 25). For some applications, computer processor 26 is configured to control apparatus 20 in response to user input received through handle 25 or other user input interfaces (such as keyboard 29).

Computer processor 26 is typically a hardware device programmed with computer program instructions to produce a special-purpose computer. For example, when programmed to perform the techniques described herein, computer processor 26 typically acts as a special-purpose, ultrasound energy application computer processor.

Apparatus 20 is configured for application of ultrasound energy to tissue of a target anatomical structure within a body of a subject. For example, apparatus 20 is configured for use with a lumen of a subject that extends from a chamber of a heart of the subject. For some applications, the chamber of the heart is an atrium of the heart and the lumen extending from the atrium is a pulmonary vein.

Typically, apparatus 20 applies the ultrasound energy to treat cardiac arrhythmias, such as atrial fibrillation. In accordance with some applications of the present invention, the ultrasound energy is applied towards myocardial tissue, and in particular towards sites within myocardial tissue which are involved in triggering, maintaining, or propagating cardiac arrhythmias, e.g., pulmonary vein ostia, and/or a mitral isthmus line. As such, as described hereinabove, apparatus 20 is shaped and sized for use at least with the pulmonary vein that extends from the left atrium of the heart. Apparatus 20 is configured to apply the ultrasound energy to cause ablation of the tissue of the pulmonary vein ostia resulting in scarring of the tissue at the ablated sites. The scars typically block abnormal electrical pulses generated in the pulmonary vein ostia from propagating into the heart chambers, thereby electrically isolating the pulmonary veins from the atrium and reducing or preventing cardiac arrhythmias.

It is noted that the scope of the present invention includes using the apparatus and methods described herein in anatomical locations other than the pulmonary vein and the left atrium (or any other cardiac structure). Therefore, apparatus 20 and/or portions thereof are sometimes referred to herein (in the specification and the claims) as an apparatus for application of the ultrasound energy to tissue of a subject. In this context, in the specification and in the claims, “lumen” refers to an inner open space, i.e., a cavity, within an organ in a subject's body, which may be, but is not necessarily, a tubular organ. For example, the ultrasound transducer may be placed within a lumen of an artery, vein, intestine, heart, stomach, bladder, sinus, lungs, lung vasculature, respiratory tract of the subject or urogenital tract of the subject.

As shown in FIG. 1, catheter 40 (which is a transluminal ablation catheter) comprises an elongated shaft having a proximal end comprising handle 25 and a distal end to which ultrasound transducer 50 is coupled. It is noted that in this context, in the specification and in the claims, “proximal” means closer to the user of the apparatus, and “distal” means farther from the user, and farther into the subject's body from the orifice through which the apparatus is originally placed into the body.

Catheter 40 facilitates advancement of ultrasound transducer 50 into the appropriate anatomy, e.g., the chamber of the heart of the subject, (e.g., the atrium), in a minimally invasive procedure. Ultrasound transducer 50 is typically inserted into the atrium to ablate tissue of the ostium of the lumen, e.g., the pulmonary vein ostium, by applying ultrasound energy to the tissue of the ostium. Additionally, or alternatively to applying ablative ultrasound energy, ultrasound transducer 50 is configured to image tissue of the subject by applying non-ablative ultrasound energy.

For some applications, ultrasound transducer 50 comprises a side-facing ultrasound transducer. For other applications, ultrasound transducer 50 comprises a distally-facing ultrasound transducer. For some applications, more than one ultrasound transducer 50 is coupled to catheter 40, with at least one distally-facing ultrasound transducer and at least one side-facing ultrasound transducer.

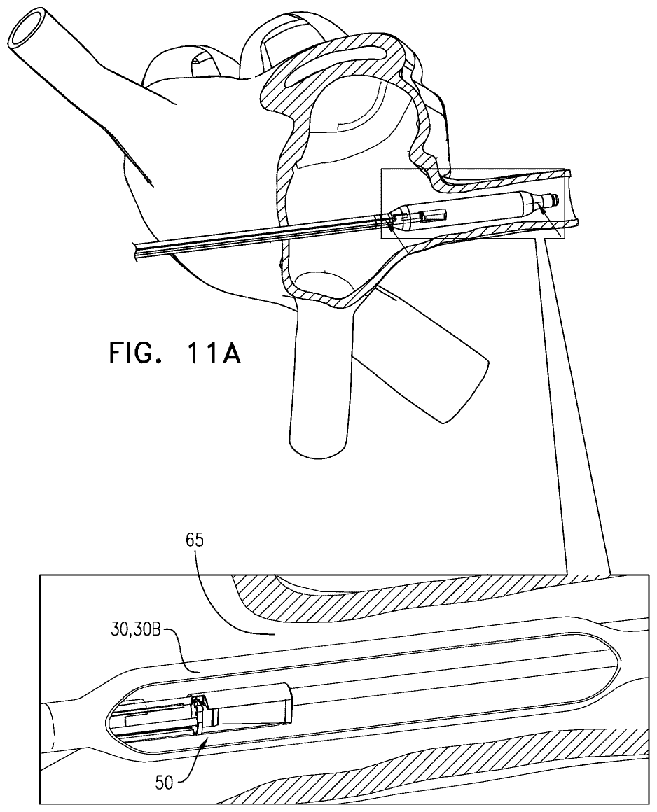

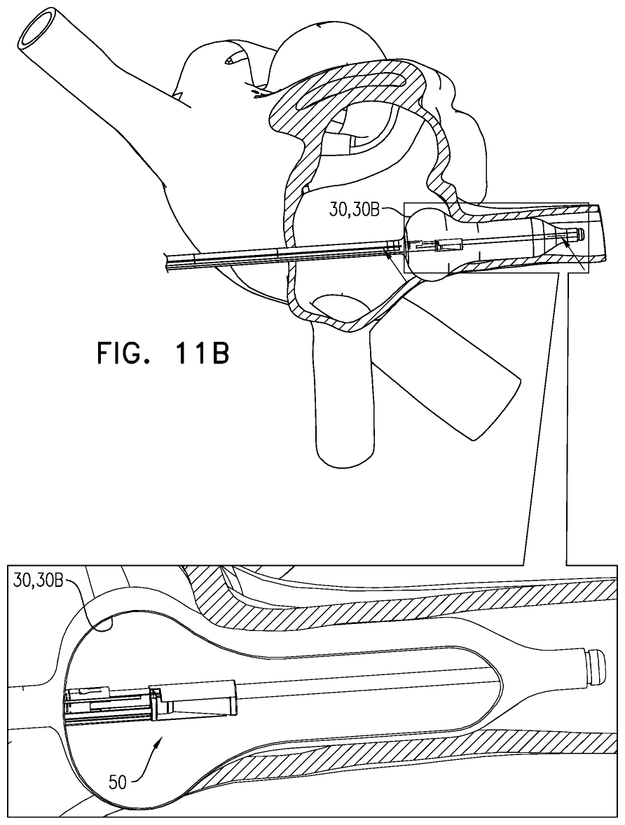

For some applications, apparatus 20 further comprises an expandable element 30 configured, inter alia, to anchor apparatus 20 in a lumen of the subject, by being disposed around ultrasound transducer 50 at the distal portion of catheter 40. For some applications, the expandable element comprises an expandable cage 30A, as shown in FIG. 1. Alternatively or additionally, for some applications, expandable element 30 comprises an inflatable element, e.g., a balloon (e.g., balloon 30B shown in FIG. 11).

Typically, ultrasound transducer 50 is configured to undergo motion. For example, ultrasound transducer is configured to undergo axial back and forth motion along a longitudinal axis LA of catheter 40. Additionally, ultrasound transducer 50 is a rotatable element. Ultrasound transducer 50 is rotatable with respect to longitudinal axis LA of catheter 40 in the direction indicated by arrow A1, in FIG. 1. Rotational and axial motion of ultrasound transducer 50 generally facilitates various functions and operational features of apparatus 20, as described in further detail hereinbelow.

For example, ultrasound transducer 50 is configured to generate an image of the tissue by applying non-ablative ultrasound energy to the tissue. For some applications, ultrasound transducer 50 generates a three-dimensional image (e.g., of the pulmonary vein ostium) by rotating around longitudinal axis LA of transluminal catheter 40, in the direction indicated by arrow A1, and longitudinally translating back and forth along longitudinal axis LA. The images that are generated may be displayed on display 23 shown in FIG. 1.

Additionally, or alternatively, ultrasound transducer 50 rotates at different rotational positions to aim transducer 50 at a tissue site that is designated for ablation/imaging. Further additionally, or alternatively, ultrasound transducer 50 may be rotated while continuously transmitting ablating ultrasound energy, thus creating a continuous circular lesion surrounding the ostium of the lumen of the blood vessel (e.g., the pulmonary vein ostium).

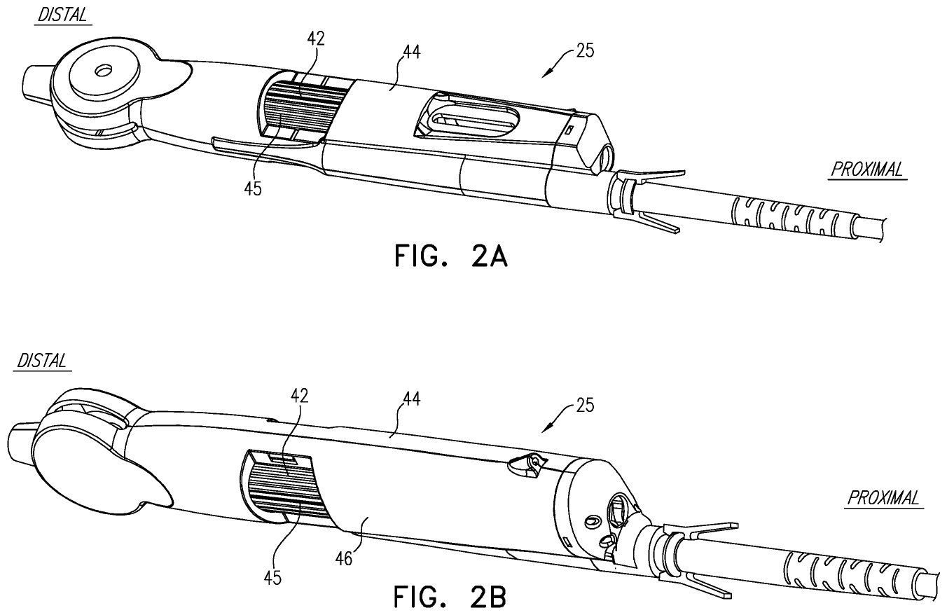

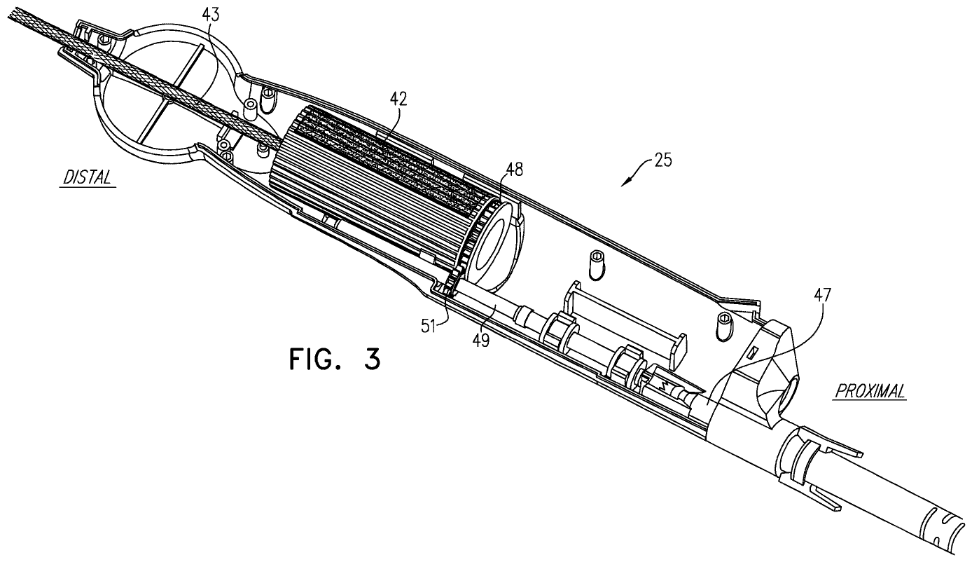

Reference is now made to FIGS. 2A, 2B and 3, which are schematic illustrations of a handle 25 of apparatus 20, in accordance with some applications of the present invention. As described hereinabove, apparatus 20 is configured to allow both rotational motion and axial back and forth motion of ultrasound transducer 50. Rotational motion of the ultrasound transducer typically facilitates both imaging of tissue and also enhanced targeting of areas in the tissue designated for ablation. In accordance with some applications of the present invention, apparatus 20 comprises a shaft and gear mechanism that allows for both manual and motorized motion of the rotatable ultrasound transducer.

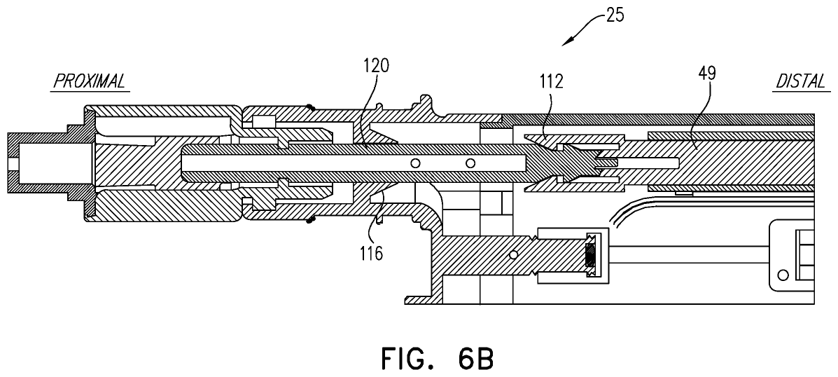

For some such applications, handle 25 at the proximal end of catheter 40 comprises a cylindrical drum 42. In general, when cylindrical drum 42 in handle 25 rotates, rotational 16 motion is transmitted from cylindrical drum 42 in handle 25 to ultrasound transducer 50 at the distal end of the catheter, via a drive cable 43. Typically, apparatus 20 is configured such that cylindrical drum 42 can be made to rotate manually by a user, or alternatively, automatically by a motor to generate automated ultrasound transducer movements. For some applications, the cylindrical drum is accessible to the user via one or more windows 45, as shown in FIGS. 2A-B, such that the user can rotate the drum directly. Alternatively, the cylindrical drum is rotated via a user-operated rotation shaft, e.g., as described in further detail hereinbelow with reference to FIGS. 6A-B.

FIGS. 2A and 2B are schematic illustrations of a view of the top portion of handle 25 having top cover 44 (FIG. 2A) and a view of the bottom portion of handle 25 having bottom cover 46 (FIG. 2B). As shown in FIGS. 2A and 2B cylindrical drum 42 is disposed in handle 25.

FIG. 3 is a schematic illustration of internal components handle 25 comprising at least a portion of a motor-coupling mechanism that facilitates both manual and motorized motion of ultrasound transducer 50, in accordance with some applications of the present invention. FIG. 3 shows cylindrical drum 42 that is configured to be rotated by a user and/or by motor 28 (motor 28 being shown in FIG. 1) via a gear wheel 48. Typically, drive cable 43 extends distally from cylindrical drum 42 to ultrasound transducer 50 at the distal end of the catheter. The drive cable transmits rotational motion from cylindrical drum 42 to the ultrasound transducer, thereby causing rotation of the ultrasound transducer. In addition, the drive cable typically transmits axial motion from cylindrical drum 42 to the ultrasound transducer, thereby causing axial motion of the ultrasound transducer.

Additionally, a handle shaft 49 is disposed within handle 25 and the handle shaft 49 extends proximally from a proximal end of cylindrical drum 42. Typically, motor 28 (shown in FIG. 1) is a component of a motor unit comprising motor 28 and a motor-unit shaft 47 extending from motor 28. The motor-unit shaft 47 is reversibly couplable to handle shaft 49, such that motor unit shaft 47 transmits rotational motion from the motor to cylindrical drum 42, when motor-unit shaft 47 is coupled to handle shaft 49 (such that rotation of cylindrical drum 42 by motor 28 causes rotation of ultrasound transducer 50). Typically, motor-unit shaft 47 additionally transmits rotational motion from the motor to cylindrical drum 42, when motor-unit shaft 47 is coupled to handle shaft 49 (such that axial motion of cylindrical drum 42 by motor 28 causes axial motion of ultrasound transducer 50). When motor-unit shaft 47 is decoupled from handle shaft 49, motor 28 does not transmit motion to the cylindrical drum, and the cylindrical drum can be rotated manually. As described hereinabove, for some applications, the cylindrical drum is accessible to the user via one or more windows 45, as shown in FIGS. 2A-B, such that manual rotation of the drum is performed directly. Alternatively, the cylindrical drum is rotated via a user-operated rotation shaft, e.g., as described in further detail hereinbelow with reference to FIGS. 6A-B.

Typically, motor 28 is configured to couple motor-unit shaft 47 and handle shaft 49 to each other by advancing a distal end of the motor-unit shaft 47 distally within handle 25, and to decouple motor-unit shaft 47 and handle shaft 49 from each other by retracting the distal end of the motor-unit shaft 47 proximally within handle 25. In such a manner, motor-unit shaft 47 is reversibly couplable to handle shaft 49 to allow motorized rotation and/or axial motion of the ultrasound transducer when motor-unit shaft 47 is coupled to handle shaft 49, and manual rotation and/or axial motion of the ultrasound transducer when motor-unit shaft 47 is not coupled to the handle shaft 49.

For some applications, apparatus 20 comprises a motor-coupling mechanism 52, which is configured to reversibly couple motor-unit shaft 47 and handle shaft 49 such that the motor unit shaft transmits rotational motion and/or axial motion from the motor to the handle shaft.

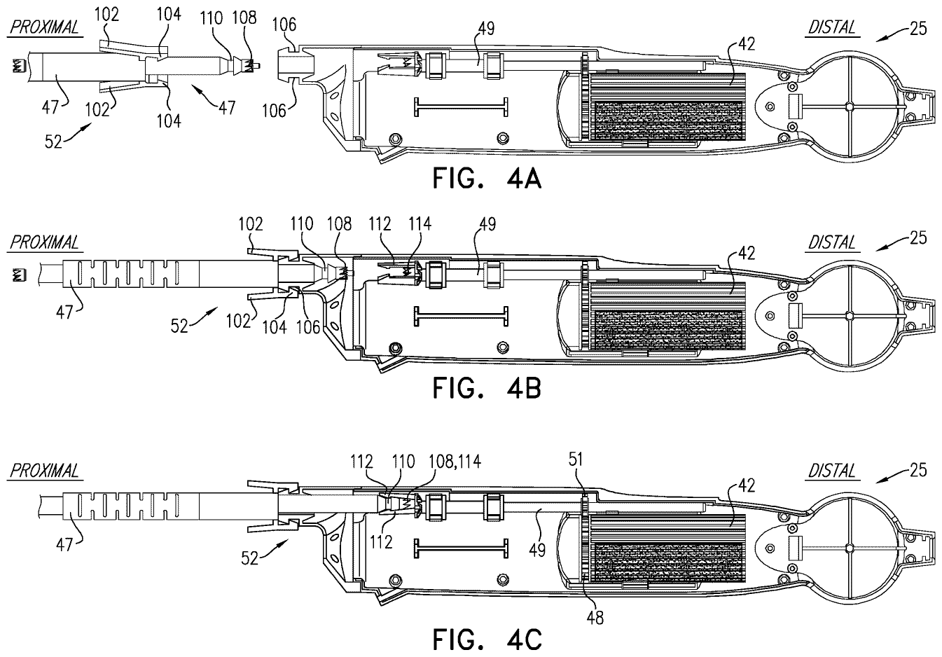

Reference is now made to FIGS. 4A-4C, which are schematic illustrations of the engagement of motor-coupling mechanism 52 configured to reversibly couple motor-unit shaft 47 and handle shaft 49 to transmit rotational motion and axial motion from the motor to the ultrasound transducer, in accordance with some applications of the present invention. Typically, motor-coupling mechanism 52 includes both an external coupling mechanism and an internal coupling mechanism. Further typically, the internal coupling mechanism couples motor-unit shaft 47 and handle shaft 49 such as to provide both rotational and axial coupling. For some applications, both the internal coupling mechanism and the external coupling mechanism are snap-coupling mechanisms.

Typically, in order to connect motor-unit shaft 47 and handle shaft 49 to transmit rotational motion and axial motion from the motor to the ultrasound transducer, the motor advances motor-unit shaft 47 towards handle 25 as shown in the transition from FIG. 4A to FIG. 4B. Typically, as motor-unit shaft 47 is advanced towards handle 25, the external coupling mechanism is effected by protrusions 104 snapping into indentations 106 in handle 25. FIG. 4B shows motor unit shaft 47 securely, but reversibly, coupled to handle 25 via the external coupling mechanism, i.e., by protrusions 104 having been placed into indentations 106. Once motor-unit shaft 47 is snapped into place with handle 25, motor-unit shaft 47 is advanced within the handle (for example, by between 20 mm and 40 mm, e.g., approximately 30 mm) to engage handle shaft 49 (FIG. 4B). For some applications, distal end of motor unit shaft 47 is shaped to define motor-unit-shaft teeth 108 and recessed neck 110. A proximal end of handle shaft 49 is shaped to define a two or more prongs and 112 and handle-shaft teeth 114 that are configured to be complementary to motor-unit-shaft teeth 108. As shown in FIG. 4C, as the motor-unit shaft 47 is advanced towards handle shaft 49, (a) an axial internal engagement mechanism is effected by prongs 112 snapping into recessed neck 110 of motor-unit shaft 47, and (b) a rotational internal engagement mechanism is effected by motor-unit-shaft teeth 108 engaging with handle-shaft teeth 114.

Typically, handle shaft 49 is both axially and rotationally engaged with drum 42. For example, as shown in FIG. 4C, a gear wheel 51 at the distal end of the handle shaft rotationally engages with gear wheel 48 at the proximal end of drum 42. For some applications, gear wheel 48 is recessed and gear wheel 51 is inserted into the recess, such that gear wheel 51 is axially engaged with drum 42. Thus, once the axial internal engagement mechanism is effected, when the motor drives the motor-unit shaft to undergo axial motion, this causes the handle shaft to undergo axial motion, which causes the drum to undergo axial motion, which in turn causes drive cable 43 to undergo axial motion. Similarly, once the rotational engagement mechanism is effected, when the motor drives the motor-unit shaft to undergo rotational motion, this causes the handle shaft to undergo rotational motion, which causes the drum to undergo rotational motion, which in turn causes drive cable 43 to undergo rotational motion.

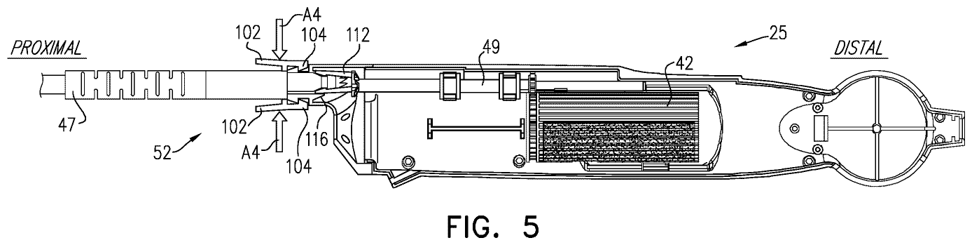

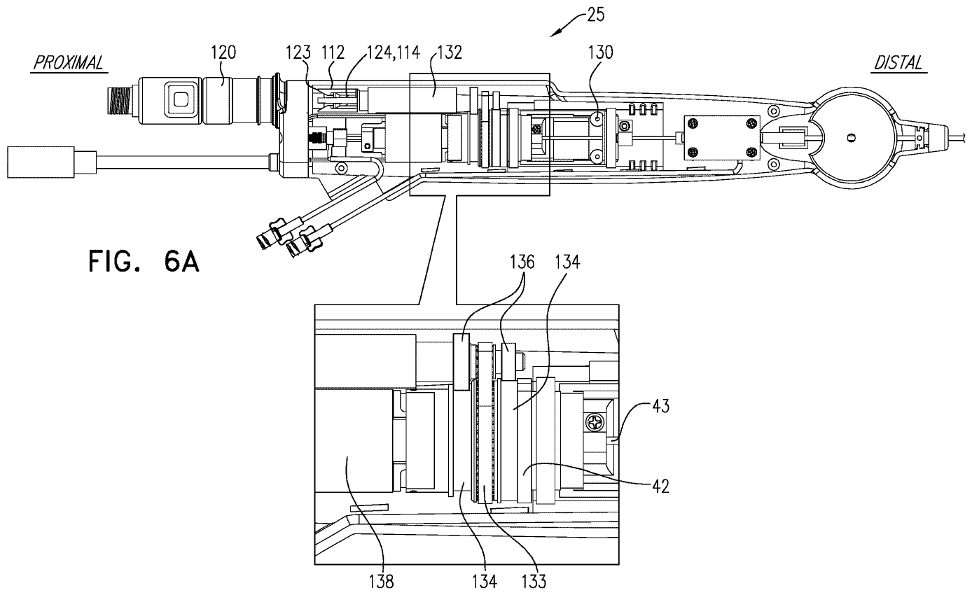

Reference is now made to FIG. 5, which is a schematic illustration of the disengagement of motor-coupling mechanism 52. For some applications, in order to decouple motor-unit shaft 47 from handle shaft 49, the motor retracts the motor-unit shaft 47 proximally. For some applications, handle 25 defines internal lips 116, as shown in FIG. 5. As prongs 112 at the proximal end of the handle shaft are pulled over the lips, the prongs are opened by internal lips 116 thereby decoupling motor-unit shaft 47 from handle shaft 49. Typically, the external coupling mechanism is released manually by a user compressing the proximal ends 102 of protrusions 104, thereby releasing protrusions 104 from indentations 106, as indicated by arrows A4 in FIG. 5.

Reference is still made to FIGS. 1-5. As described hereinabove, ultrasound transducer 50 is capable of both rotational and linear motion. Ultrasound transducer 50 is typically configured to apply a treatment to (and/or image tissue of) the subject when the ultrasound transducer is disposed at a plurality of different rotational positions. As described hereinabove, the drive cable, which extends distally from within the handle to the ultrasound transducer at the distal end of the catheter, typically transmits rotational and axial motion from within the handle to the ultrasound transducer. In some cases, static friction occurs while the ultrasound transducer undergoes rotational movements between the plurality of different rotational positions, causing a delay in rotation. Typically, motor 28 is configured, during the treatment, to drive the ultrasound transducer 50 to undergo rotational movements between the plurality of different rotational positions, and to reduce static friction acting upon the rotatable element between the rotational movements, by driving the ultrasound transducer to continuously move axially between the rotational movements of the ultrasound transducer. Typically, relatively small axial back-and-forth motions are applied between the rotational movements. In this manner, the transducer and the drive cable are not susceptible to static friction, which would occur if the drive cable and the transducer were entirely stationary between the rotational movements. Typically, the axial motion is applied by means of relatively small axial back-and-forth motions during the rotational movement. Typically, this generates constant dynamic friction, rather than the static friction associated with stepwise rotational movements, which is typically higher.

Reference is now made to FIGS. 6A and 6B, which are schematic illustrations of user-operated rotation shaft 120 for coupling to handle 25 of the apparatus to transmit rotational motion and axial motion from a human operator to the ultrasound transducer, in accordance with some applications of the present invention. As described hereinabove, for some applications, the cylindrical drum is accessible to the user via one or more windows 45, as shown in FIGS. 2A-B, such that manual rotation of the drum is performed directly. Alternatively, the cylindrical drum is rotated via a user-operated rotation shaft 120. Typically, the user-operated rotation shaft is coupled to the handle shaft 49 in a generally similar manner to that described hereinabove with reference to the motor-unit shaft 47. For some applications, the user-operated rotation shaft is advanced within the handle by the user to engage handle shaft 49. For some applications, the distal end of user-operated rotation shaft 120 is shaped to define user-operated rotation shaft teeth 124 and recessed neck 123. As described hereinabove, the proximal end of handle shaft 49 is shaped to define a two or more prongs 112 and handle-shaft teeth 114. Typically, handle-shaft teeth 114 are configured to be complementary to user-operated rotation shaft teeth 124. As shown in FIG. 6A, as the user-operated rotation shaft is advanced towards handle shaft 49, (a) an axial internal engagement mechanism is effected by prongs 112 snapping into recessed neck 123 of user-operated rotation shaft 120, and (b) a rotational internal engagement mechanism is effected by user-operated rotation shaft teeth 124 engaging with handle-shaft teeth 114.

Still referring to FIG. 6A, for some applications an accelerometer 130 is disposed within handle 25 and is configured to detect roll angular rotation of drive cable 43. For some applications, the handle shaft is covered with a friction-reducing sleeve 132, for example a polytetrafluoroethylene sleeve that is configured to reduce friction between the handle shaft and the handle housing. Typically, handle shaft 49 is both axially and rotationally engaged with drum 42. For some applications, different engagement mechanisms to those described with reference to FIG. 4C are used. For example, as shown in FIG. 6A, drive belt 133 rotationally engages with drum 42. For some applications, the drum includes recesses 134 and there are bearings 136 disposed around the handle shaft that are disposed within the recesses. Typically, the handle shaft is axially engaged with the drum (and thereby axially engaged with the drive cable) via the bearing being inserted within the recesses of the drum. For some applications, a slip ring 138 is disposed within the handle, thereby allowing rotation of electrical wires without them becoming twisted with each other.

Referring to FIG. 6B, as described herein above with reference to motor-unit shaft 47, user-operated rotation shaft 120 is typically decoupled from handle shaft 49 by retracting the user-operated rotation shaft proximally. For some applications, handle 25 defines internal lips 116. As prongs 112 at the proximal end of the handle shaft are pulled over the lips, the prongs are opened thereby decoupling user-operated rotation shaft 120 from handle shaft 49.

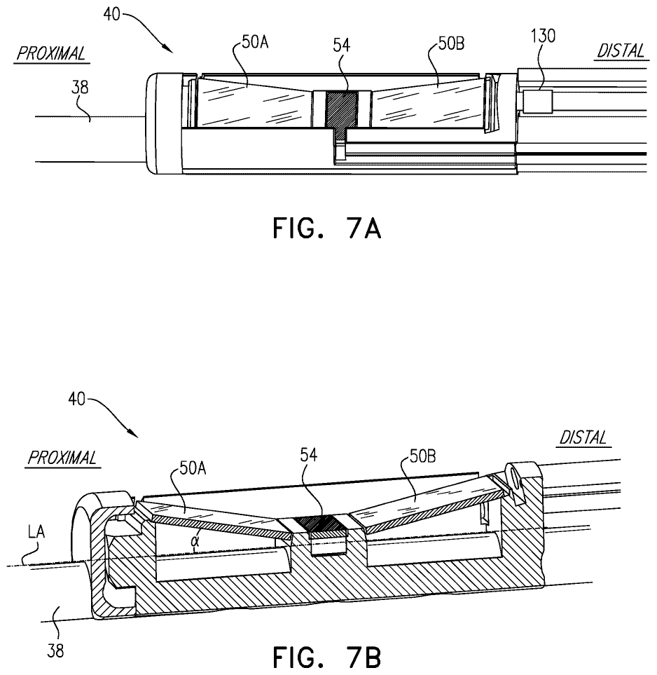

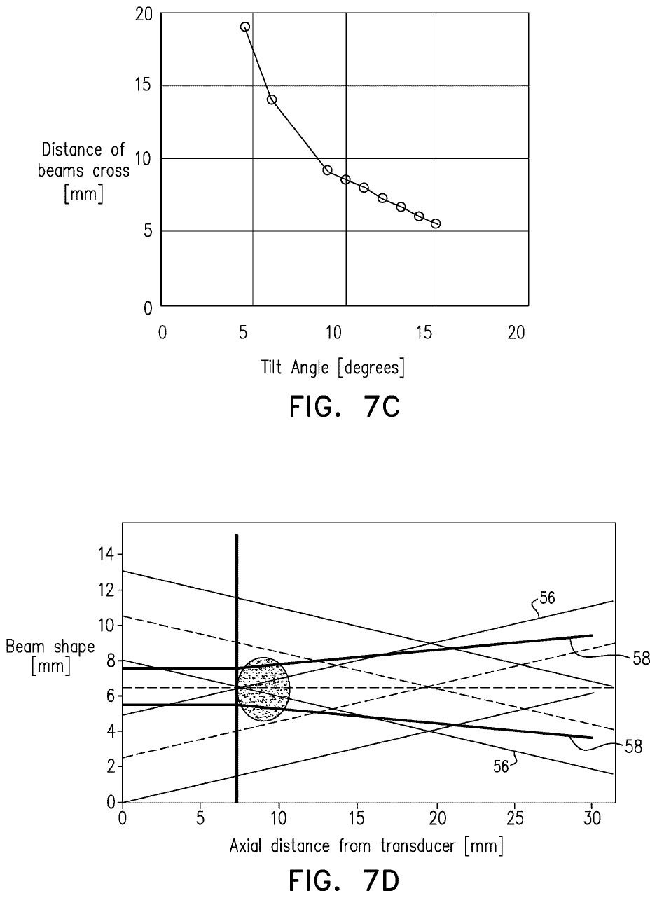

Reference is now made to FIGS. 7A and 7B, which are schematic illustrations of a configuration of ultrasound transducers in apparatus 20, in accordance with some applications of the present invention.

In accordance with some applications of the present invention, in addition to applying ultrasound energy for ablation purposes via ultrasound transducer 50, apparatus 20 additionally comprises an additional ultrasound transducer that transmits non ablating ultrasound energy. For example, the non-ablating ultrasound transducer is configured to perform acoustic sensing. For some applications, the acoustic sensing is performed by transmitting one or more pulses of pulse-echo ultrasound energy towards the designated tissue site and receiving a reflection of the transmitted pulse-echo ultrasound energy. Generally, a parameter of the reflected pulse-echo ultrasound energy can be indicative of the ultrasound energy applied to the tissue and of an effect of the ultrasound energy on the tissue. Additionally, or alternatively, the reflected ultrasound energy can serve as input for altering operational parameters of the ultrasound transducers, for example, the power, duty cycle, or any other parameter of the ablation are modulated in response to the detected reflected ultrasound energy by the non-ablating ultrasound transducer. For some applications, the reflected ultrasound energy is used to assess the outcome of an ablation treatment post-treatment, and/or to assess lesion formation progression during an ablation treatment. Additionally, or alternatively, the non-ablating ultrasound transducer is configured to image tissue of the subject by applying non-ablative ultrasound energy.

For some such applications, apparatus 20 comprises more than one, e.g., two ablative ultrasound transducers 50A and 50B configured to be inserted into the chamber of the subject's heart, and to ablate tissue of an ostium of the lumen by applying ablative ultrasound energy to the tissue of the ostium. Typically, each of ablative ultrasound transducers 50A and 50B are positioned such that the ultrasound beams emitted from ultrasound transducers 50A and 50B converge to a converging ultrasound beam configured to focus on the target tissue.

For some applications, as shown in FIGS. 7A and 7B, ultrasound transducers 50A and 50B are disposed on a side of shaft 38 of catheter 40 at an angle alpha of between 8 degrees and 15 degrees, e.g., 11.5 degrees, from axis LA of shaft 38 of catheter 40, such as to create a convergent ultrasound beam from the beams emitting from each of transducers 50A and 50B. The convergent ultrasound beam typically increases the ultrasound energy that is focused on the target tissue. Additionally, sensing ultrasound transducer 54, e.g., an imaging ultrasound transducer 54, is disposed between the two ablative ultrasound transducers 50A and 50B and is configured to acquire one or more ultrasonic images of the tissue of the target tissue (e.g., the ostium of the pulmonary vein).

Reference is still made to FIG. 7A. For some applications, additionally, or alternatively to accelerometer 130 in handle 25, apparatus 20 comprises an accelerometer 130 that is disposed in proximity to the ultrasound transducer within the distal portion of the catheter. Accelerometer 130 is configured to detect rotation at the exact position of the transducer.

Reference is made to FIG. 7C, which is a graph showing the axial distance from ablative ultrasound transducers 50A and 50B at which the beams from these ultrasound transducers cross as a function of the tilt angle of the ultrasound transducers, in accordance with some applications of the present invention. As described hereinabove, and as shown in FIGS. 7A and 7B, ultrasound transducers 50A and 50B are disposed on a side of shaft 38 of catheter 40 at an angle alpha of between 8 degrees and 15 degrees from the axis of shaft 38 of catheter 40. This titling of the transducers causes the beams 56 (indicated in FIG. 7D) that are emitted from ablative transducers 50A and 50B to converge and cross at distances that are affected by the angle at which the transducers are tilted.

FIG. 7D is a graph showing the beam shape and axial distance from ultrasound transducers 50A and 50B at which the beams cross for the respective tilt angles of the ultrasound transducers shown in FIG. 7C, in accordance with some applications of the present invention. As shown, when the ultrasound transducers 50A and 50B are tilted such as to be disposed on the side of shaft 38 of catheter 40 at an angle of 11.5 degrees, beams 56 cross at a distance of about 7.5 mm, e.g., 7.3 mm, from the transducers. As described hereinabove, an imaging transducer 54 is disposed between ablative transducers 50A and 50B. The beams 58 emitted from imaging transducer 54 are indicated in the graph shown in FIG. 7D.

Typically, a distance of ultrasound transducers 50A and 50B from the ablation site is 4-8 mm, e.g., 4 mm. Additionally, or alternatively, a depth of the transmural lesion caused by ablation with ultrasound transducers 50A and 50B is 1-5 mm, e.g., 3.5 mm.

For some applications ablative ultrasound transducers 50A and 50B are configured to transmit the ultrasound energy to ablate the tissue at a frequency of 8-20 MHz, e.g., 10-12 MHz, e.g., 11 MHz, and at a power level of more than 3 W, e.g., 3 W-50 W, e.g., 6-35 W. For some applications, non-ablating ultrasound transducer 54 is configured to transmit the ultrasound energy at a frequency of 5-60 MHz power level of up to (e.g., less than) 2 W.

For some applications, the surface area of each of ablative transducers 50A and 50B is between 5 mm\hat{ }2-20 mm\hat{ }2 and for example 12 mm\hat{ }2. For some applications, a diameter of the non-ablating transducer 54 is between 1-3 mm, e.g., 2 mm. Typically, the distance between the piezoelectric element (PZT) of the ultrasound transducers is between 0.1-1 mm, e.g., 0.5 mm.

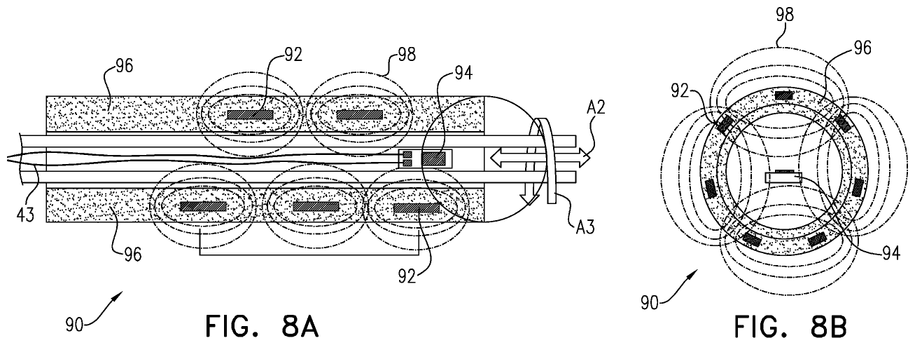

Reference is now made to FIGS. 8A and 8B, which are schematic illustrations of a mechanism configured to determine the rotational and axial position of ultrasound transducer 50, in accordance with some applications of the present invention. As described hereinabove, apparatus 20 is configured to allow both rotational motion (indicated by arrow A3 in FIG. 8A) and axial back and forth motion (indicated by arrow A2 in FIG. 8A) of the ultrasound transducer(s). It is generally desirable to accurately determine axial and/or rotational positions of the ultrasound transducer(s) that undergo motion. FIGS. 8A and 8B, are schematic illustrations of a sensor mechanism configured to determine the rotational and/or axial position of ultrasound transducer 50 (e.g., transducer 50A and/or 50B), in accordance with some applications of the present invention. More specifically, FIGS. 8A and 8B are schematic illustrations of cross sections of apparatus 20 comprising a magnetic sensor mechanism 90, in accordance with some applications of the present invention. Magnetic sensor mechanism 90 typically comprises a plurality of passive magnetic rods 92 and an active magnetic sensor 94.

As described hereinabove, the ultrasound transducer is disposed at the distal end of the catheter, and rotational and axial motion is transmitted to the ultrasound transducer through drive cable 43 extending distally from within the handle to the ultrasound transducer at the distal end of the catheter. Typically, a stationary tube 96 is disposed around drive cable 43, the tube being configured to remain stationary as the drive cable undergoes rotational and axial motion. A plurality of magnetic rods 92 are disposed a respective axial and rotational locations within a given region of stationary tube 96, each of magnetic rods 92 are configured to generate magnetic fields 98 having respective, different characteristics from each other, for example, by having different lengths, diameters, thicknesses, and/or magnetic properties from each other. Magnetic sensor 94 is typically coupled to drive cable 43 and is configured to detect the distinct magnetic fields generated by magnetic rods 92. Computer processor 26 typically receives a signal generated by magnetic sensor 94 (in response to detecting the different magnetic fields generated by magnetic rods 92) and derives axial and rotational positions of the ultrasound sensor based upon the signal generated by magnetic sensor 94.

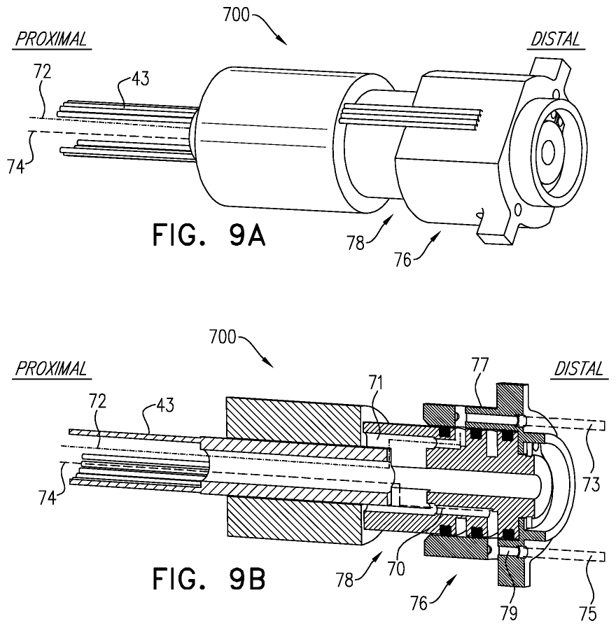

Reference is now made to FIGS. 9A and 9B, which are schematic illustrations of a fluid lumen rotational manifold, in accordance with some applications of the present invention. As described hereinabove, catheter 40 typically comprises rotatable ultrasound transducer 50 at the distal end of the catheter. Catheter 40 typically comprises fluid lumens for delivering fluid to the vicinity of the rotatable ultrasound transducer for cooling of the ultrasound transducer. For some such applications, catheter 40 comprises a fluid lumen rotational manifold 700 configured to prevent rotation of fluid lumens while the ultrasound transducer rotates, thereby preventing twisting and tangling of the fluid lumens. Typically, drive cable 43 that extends distally from within the handle to the rotatable ultrasound transducer (the drive cable transmits rotational motion from within the handle to the rotatable ultrasound transducer), defines a drive-cable fluid inflow lumen 72 and a drive-cable fluid outflow lumen 74 for cooling of the rotatable ultrasound transducer.

Fluid manifold 700 typically includes a fluid entry port 73, a fluid exit port 75, a rotationally static portion 76 that contains a rotationally-static fluid inflow lumen 77, which is in fluid communication with fluid entry port 73, and a rotationally-static fluid outflow lumen 79, which is in fluid communication with fluid exit port 75. The fluid manifold additionally comprises a rotational portion 78 that is configured to rotate with drive cable 43. Rotational portion 78 and defines (a) a rotational fluid inflow lumen 71 that is in fluid communication with both the rotationally-static fluid inflow lumen 77 and the drive-cable fluid inflow lumen 72, thereby placing rotationally-static fluid inflow lumen 77 and the drive-cable fluid inflow lumen 72 in fluid communication with each other; and (b) a rotational fluid outflow lumen 70 that is in fluid communication with rotationally-static fluid outflow lumen 79 and the drive-cable fluid outflow lumen 74, thereby placing the rotationally-static fluid outflow lumen and the drive-cable fluid outflow lumen in fluid communication with each other. In such a manner, apparatus 20 is configured to provide fluid cooling of the rotatable ultrasound transducer, while preventing rotation and consequent twisting of the fluid lumens.

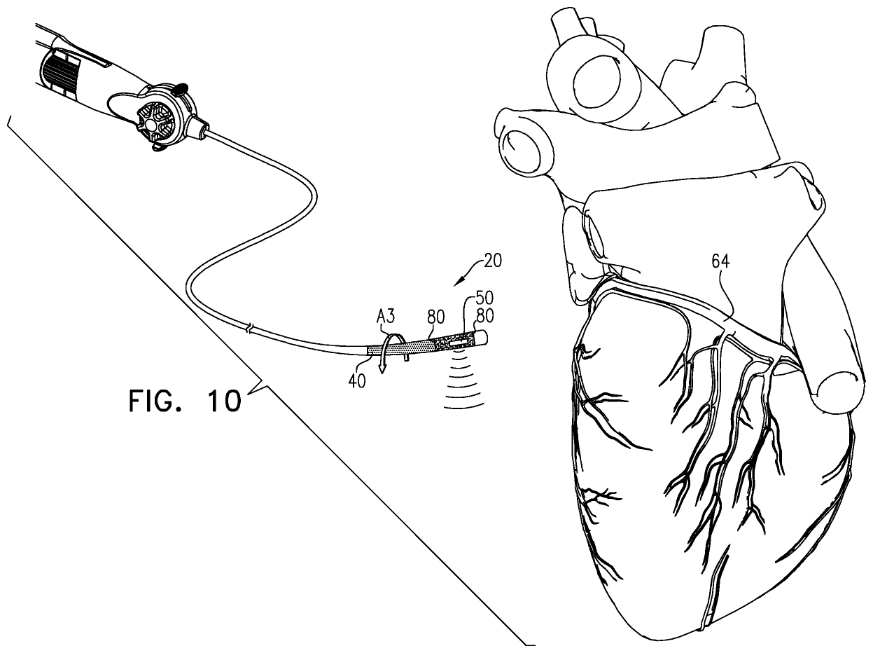

Reference is now made to FIG. 10, which is a schematic illustration of apparatus 20 for application of ultrasound energy to tissue within the body of a subject, in accordance with some applications of the present invention. Typically, as described hereinabove, apparatus 20 is configured to be positioned within a lumen in a body of a subject and to apply ablative energy in order to ablate tissue surrounding the lumen. In some cases, apparatus 20 is positioned and activated to ablate the ostium of the pulmonary vein that extends from the left atrium of the heart. Typically, in such cases apparatus 20 applies the ultrasound energy to ablate a circumferential lesion in the ostium of the pulmonary vein in order to electrically isolate the pulmonary vein to treat cardiac arrhythmia by disrupting the conduction of abnormal electrical impulses.

In accordance with some applications of the present invention, use of apparatus 20 is not limited to ablation of blood vessel orifices such as the pulmonary vein ostium. Rather, apparatus 20 is configured to be applied to any region in the heart that is involved in triggering or maintaining cardiac arrhythmias.

For example, in some subjects, atrial fibrillation persists even after pulmonary vein isolation due to abnormal electrical impulses originating from the mitral isthmus. For some applications of the present invention, apparatus 20 is configured to be positioned and activated to ablate a linear lesion in the mitral isthmus (and/or in the cavo-tricuspid isthmus line, as described hereinbelow), to treat atrial fibrillation and/or atrial flutters in addition to (or alternatively to) ablation of the pulmonary vein ostium. Typically, apparatus 20 is configured to apply ablative energy to ablate the mitral isthmus from within coronary sinus 64 to create an ablation line between the left inferior pulmonary vein (LIPV) to the lateral mitral annulus. In accordance with some applications of the present invention, catheter 40 is inserted into the coronary sinus and transducer 50 is activated to ablate a typically thick transmural lesion spanning the entire myocardial thickness and reaching the mitral isthmus. For some applications, the ablative ultrasound energy is applied in the coronary sinus as a directional, collimated beam to form a linear lesion from the mitral isthmus to the left inferior pulmonary vein. Use of a highly directional and collimated beam of ablative ultrasound energy transmitted by apparatus 20 in accordance with applications of the present invention, allows safe and targeted application of the energy to create a linear lesion from the mitral isthmus to the left inferior pulmonary vein, while avoiding potential damage to cardiac structures including blood vessels. This is in contrast to the application of other forms of energy which may scatter and cause unintentional damage to surrounding tissue.

For some applications, ablation from the mitral isthmus to the left inferior pulmonary vein is accompanied by imaging using ultrasound transducer 50 (or additional/alternative ultrasound transducers coupled to catheter 40). The imaging typically includes brightness imaging (B mode) and/or motion imaging (M-mode). The imaging is typically utilized to identify, and thereby avoid, critical blood vessels that may be located in the line of the ablative ultrasound energy, thereby avoiding potential damage to the blood vessels. Additionally, or alternatively, the imaging capabilities of apparatus 20 facilitate proper identifying of the mitral isthmus which is the target area in the tissue.

Additionally, or alternatively, apparatus 20 is configured to apply ablative energy to the cavo-tricuspid isthmus line (CTI line) to treat atrial flutters by ablating a linear lesion between the inferior vena cava (IVC) and the tricuspid valve.

As shown in FIG. 10, for some applications, catheter 40 comprises at least two electrodes 80 coupled to the distal end of the catheter and positioned on either side of ultrasound transducer 50. Electrodes 80 are configured to measure electrical potential between electrodes 80 to thereby identify whether catheter 40 is located in the vicinity of an anatomical region that causes abnormal electrical impulses that can be terminated by ablation thereof.

Reference is still made to FIG. 10. In accordance with some applications of the present invention, coronary sinus ablation using apparatus 20 is configured to be applied for treatment of premature ventricular contractions (PVCs) that typically originate from the left ventricular summit.

Generally, ablation of ventricular arrhythmias or premature ventricular contractions (PVCs) originating from the left ventricular (LV) summit remain a challenge because of anatomical constraints, including myocardial thickness and the availability of access (proximity to major coronary arteries, thick epicardium in an epicardial approach). Also, ablating from the outside of the heart is particularly difficult due to layers of fat that are disposed on the outside of the heart and may be located in the line of the ablative ultrasound energy directed at the left ventricular (LV) summit. Additionally, ablating the left ventricular (LV) summit from the inside of the heart also poses difficulties.

In accordance with some applications of the present invention, apparatus 20 is configured for ablation of left ventricular (LV) summit from outside the heart when catheter 40 is positioned within coronary sinus 64. Typically, for such applications, apparatus 20 is advanced through coronary sinus 64 to the great cardiac vein (GCV). From the great cardiac vein (GCV), transducer 50 is aimed to form an ablative lesion at the (LV) summit to treat premature ventricular contractions (PVCs). As described hereinabove, electrodes 80 are configured to measure electrical potential between electrodes 80 to thereby identify whether catheter 40 is located in a position the vicinity of an anatomical region that causes abnormal electrical impulses that can be terminated by ablation thereof. Additionally, or alternatively, B mode and/or M-mode ultrasound imaging with ultrasound transducer 50 (or additional/alternative ultrasound transducers coupled to catheter 40), is utilized to identify, and thereby avoid, critical blood vessels that may be located in the line of the ablative ultrasound energy, thereby avoiding potential damage to the blood vessels. Additionally, or alternatively, the imaging capabilities of apparatus 20 facilitate proper identifying of the left ventricular (LV) summit which is the target area in the tissue.

Reference is now made to FIGS. 11A-11B, which are schematic illustrations of apparatus 20 comprising an expandable element 30 for application of ultrasound energy to tissue within a body of a subject, in accordance with some applications of the present invention. As described hereinabove with reference to FIG. 1, for some applications, apparatus 20 comprises an expandable element 30 configured, inter alia, to anchor apparatus 20 in a lumen of the subject by being disposed around ultrasound transducer 50 at the distal portion of catheter 40. Typically, expandable element 30 positions and temporarily anchors the distal portion of catheter 40 in a lumen e.g., the pulmonary vein, by contacting a wall of the lumen. For some applications, as shown in FIG. 1, expandable element 30 comprises an expandable cage 30A.

Alternatively, for some applications, expandable element 30 comprises an inflatable element, e.g., balloon 30B, as shown in FIGS. 11A-11B. For some applications, the balloon is a non-compliant balloon. Alternatively, balloon 30B comprises a compliant balloon having a cylindrical shape and configured to be inflated to various diameters rending it suitable for insertion into anatomical structures of various shapes and sizes for treatment of a variety of cardiac arrhythmias, such as atrial fibrillation, atrial flutters, and/or atrial tachycardia. For example, balloon 30B is a highly compliant cylindrical balloon that is configured to change in diameter from a diameter that is suitable for insertion into pulmonary vein 65, (e.g., a diameter of 8 mm-30 mm), to a smaller diameter (e.g., 4 mm-10 mm) that is suitable for insertion into narrow and elongated anatomical structures, such as coronary sinus 64 (shown in FIG. 10). Balloon 30B is additionally configured for positioning of apparatus 20 in location that allows for ablation of linear lesions of the cavo-tricuspid isthmus line (CTI line), and for ablation of linear lesions within the left atrium between the four pulmonary veins.

Typically, balloon 30B, which is compliant and shaped and sized to define a cylindrical balloon (resembling a hotdog), is configured to position apparatus 20 in the pulmonary vein to allow for ablation by apparatus 20 of a circular lesion in the pulmonary vein ostium (and subsequent touch-up lesions at locations in the pulmonary veins that still trigger cardiac arrhythmias following creation of the circular lesion). Additionally, balloon 30B is configured, to position apparatus 20, e.g., in the coronary sinus, for ablating linear lesions. Typically, due to its compliant nature, balloon 30B has elongation parameters of up to 400% along the circumferential direction such that it is highly compliant as it expands radially. For some applications, balloon 30B is configured to vary in diameter along a length of the ballon, so as to fit changes within the anatomical structure.

Typically, the balloon (or a different expandable element) is configured to facilitate the formation of a linear lesion within a lumen in which it is placed by allowing the ultrasound transducer to be moved axially within the balloon while the ballon is in an axially fixed in position with respect to the wall of the lumen and the ultrasound transducer is applying ultrasound energy towards the tissue. Alternatively, the balloon (or a different expandable element) is configured to facilitate the formation of a linear lesion within the lumen by the entire catheter being pulled along the lumen with the balloon in a partially inflated (or expanded) state (such that the ballon is not held is in an axially fixed in position with respect to the wall of the lumen), while the ultrasound transducer is applying ultrasound energy towards the tissue.

Reference is again made to FIGS. 1-11. It is noted that in accordance with some applications of the present invention, apparatus 20 can be used to treat types of cardiac arrhythmia other than atrial fibrillation. For example, apparatus 20 is used to treat conditions such as ventricular tachycardia. For such applications, apparatus 20 is advanced into a ventricle of a subject and lesions are created by ablation of tissue in the ventricle by application of ultrasound energy in accordance with applications of the present invention.

It is further noted that application of ultrasound energy to myocardial sites is not limited to blood vessel orifices but may be applied to any region in the heart which is involved in triggering or maintaining cardiac arrhythmias.

It is further noted that, although much of the description herein relates to cardiac tissue, particularly, the left atrium and pulmonary veins extending from the atrium, the scope of the present invention includes the use of the apparatus and methods described herein with respect to other lumens in the body (“lumen” generally referring to an inner open space, i.e., a cavity, within an organ in a subject's body, which may be, but is not necessarily, a tubular organ). For example, the apparatus and methods described herein may be used, mutatis mutandis, with respect to an artery, vein, intestine, heart, bladder, sinus, stomach, lungs, lung vasculature, respiratory tract of the subject or urogenital tract of the subject.

It is further noted that apparatus and methods described herein may additionally be used, mutatis mutandis, to treat other tissue of a subject for a treatment that includes renal denervation, targeted lung denervation, pulmonary hypertension denervation, splanchnic nerve denervation, carotid body denervation, cancerous lung nodule ablation, hypertrophic cardiomyopathy ablation, and/or hepatic artery denervation.

For some applications, the ultrasound energy application techniques described herein are practiced in combination with other types of ablation, such as Pulsed Field Ablation (PFA) and/or radiofrequency (RF) ablation. For some applications, other suitable energy sources (e.g., RF, laser, cryogenic, and/or electromagnetic energy such as ultraviolet and/or infrared) are used as an alternative or in addition to ultrasound ablation.

It will be appreciated by persons skilled in the art that the present invention is not limited to what has been particularly shown and described hereinabove. Rather, the scope of the present invention includes both combinations and subcombinations of the various features described hereinabove, as well as variations and modifications thereof that are not in the prior art, which would occur to persons skilled in the art upon reading the foregoing description.

1. An apparatus comprising:
a catheter having a distal end and a proximal end, the catheter comprising:
a rotatable element at the distal end of the catheter;
a handle at the proximal end of the catheter, the handle comprising a cylindrical drum;
a drive cable extending distally from the cylindrical drum to the rotatable element at the distal end of the catheter, the drive cable being configured to transmit rotational motion from the cylindrical drum to the rotatable element; and
a handle shaft disposed within the handle, the handle shaft extending proximally from a proximal end of the cylindrical drum; and
a motor unit comprising:
a motor; and
a motor-unit shaft extending from the motor, the motor-unit shaft being configured to be reversibly couplable to the handle shaft, such that the motor unit shaft transmits rotational motion from the motor to the cylindrical drum.

2. The apparatus according to claim 1, wherein the handle defines one or more windows and wherein the cylindrical drum is configured to be manually rotated directly via the one or more windows.

3. The apparatus according to claim 1, further comprising a user-operated rotation shaft configured to be couplable to the handle shaft, such as to facilitate manual rotation of the cylindrical drum, via the user-operated rotation shaft and the handle shaft.

4. The apparatus according to claim 1, wherein the catheter comprises a transluminal ablation catheter.

5. The apparatus according to claim 1, wherein the catheter comprises one or more sensors configured to detect a rotational position of the rotatable element.

6. The apparatus according to claim 1, wherein the rotatable element comprises a rotatable ultrasound transducer.

7. The apparatus according to claim 6, wherein the rotatable ultrasound transducer is configured to be inserted into a left atrium in a vicinity of a pulmonary vein ostium and is configured to ablate tissue of the pulmonary vein ostium, to thereby electrically isolate the pulmonary vein.

8. The apparatus according to claim 6, wherein the rotatable ultrasound transducer is configured to be inserted into a coronary sinus in a vicinity of a mitral isthmus and is configured to ablate tissue of the isthmus.

9. The apparatus according to claim 1, wherein the motor-unit shaft and the handle shaft comprise a snap-coupling mechanism, which is configured to reversibly couple the motor-unit shaft and the handle shaft such that the motor unit shaft transmits rotational motion and axial motion from the motor to the handle shaft.

10. The apparatus according to claim 9, wherein the motor is configured to:
couple the motor-unit shaft and the handle shaft to each other by advancing a distal end of the motor-unit shaft distally within the handle, and
decouple the motor-unit shaft and the handle shaft from each other by retracting the distal end of the motor-unit shaft proximally within the handle.

11-17. (canceled)

18. An apparatus comprising:
a catheter having a distal end and a proximal end, the catheter comprising:
a rotatable element at the distal end of the catheter configured to apply a treatment to a subject when disposed at a plurality of different rotational positions;
a handle at the proximal end of the catheter; and
a drive cable extending distally from within the handle to the rotatable element at the distal end of the catheter, the drive cable being configured to transmit rotational motion from within the handle to the rotatable element; and
a motor configured, during the treatment, to drive the rotatable element to undergo rotational movements between the plurality of different rotational positions, and to reduce static friction acting upon the rotatable element between the rotational movements, by driving the rotatable element to continuously move axially between the rotational movements of the rotatable element.

19. The apparatus according to claim 18, wherein the catheter comprises a transluminal ablation catheter.

20. The apparatus according to claim 18, the motor is configured to drive the rotatable element to continuously move axially between the rotational movements of the rotatable element, to move in an axial back and forth motion to prevent the rotatable element from being susceptible to static friction between the rotational movements of the rotatable element.

21. The apparatus according to claim 18, wherein the rotatable element comprises a rotatable ultrasound transducer.

22. The apparatus according to claim 21, wherein the rotatable ultrasound transducer is configured to be inserted into a left atrium in a vicinity of a pulmonary vein ostium and is configured to ablate tissue of the pulmonary vein ostium, to thereby electrically isolate the pulmonary vein.

23. The apparatus according to claim 21, wherein the rotatable ultrasound transducer is configured to be inserted into a coronary sinus in a vicinity of a mitral isthmus and is configured to ablate tissue of the isthmus.

24. An apparatus for use with a fluid, the apparatus comprising:
a catheter having a distal end and a proximal end, the catheter comprising:
a rotatable element at the distal end of the catheter configured to apply a treatment to a subject when disposed at a plurality of different rotational positions;
a handle at the proximal end of the catheter; and
a drive cable extending distally from within the handle to the rotatable element at the distal end of the catheter, the drive cable being configured to transmit rotational motion from within the handle to the rotatable element, the drive-cable defining a drive-cable fluid inflow lumen and a drive-cable fluid outflow lumen; and
a fluid manifold, comprising:
a fluid entry port;
a fluid exit port;
a rotationally static portion that contains a rotationally-static fluid inflow lumen, which is in fluid communication with the fluid entry port, and a rotationally-static fluid outflow lumen, which is in fluid communication with the fluid exit port; and
a rotational portion that is configured to rotate with the drive cable, the rotational portion defining:
a rotational fluid inflow lumen that is in fluid communication with both the rotationally-static fluid inflow lumen and the drive-cable fluid inflow lumen, thereby placing the rotationally-static fluid inflow lumen and the drive-cable fluid inflow lumen in fluid communication with each other; and
a rotational fluid outflow lumen that is in fluid communication with the rotationally-static fluid outflow lumen and the drive-cable fluid outflow lumen, thereby placing the rotationally-static fluid outflow lumen and the drive-cable fluid outflow lumen in fluid communication with each other.

25. (canceled)

26. The apparatus according to claim 24, wherein the rotatable element comprises a rotatable ultrasound transducer.

27. The apparatus according to claim 26, wherein the rotatable ultrasound transducer is configured to be inserted into a left atrium in a vicinity of a pulmonary vein ostium and is configured to ablate tissue of the pulmonary vein ostium, to thereby electrically isolate the pulmonary vein.

28. The apparatus according to claim 26, wherein the rotatable ultrasound transducer is configured to be inserted into a coronary sinus in a vicinity of a mitral isthmus and is configured to ablate tissue of the isthmus.

29-41. (canceled)
